(19) United States
(12) Patent Application Publication
(10) Pub. No.: US 2019/0083466 A1
(43) Pub. Date: Mar. 21, 2019

(54) NOVEL USE OF BIOTIN AND NATURAL ESSENTIAL OILS FOR BOVINE ANIMALS TO IMPROVE BEEF QUALITY

(71) Applicant: DSM IP ASSETS B.V., Heerlen (NL)

(72) Inventors: Irmgard IMMIG, Kaiseraugst (CH); Wolfgang STEINBERG, Kaiseraugst (CH); Joseph MC GRATH, Kaiseraugst (CH)

(21) Appl. No.: 16/194,652

(22) Filed: Nov. 19, 2018

Related U.S. Application Data
(63) Continuation of application No. 15/102,967, filed on Jun. 9, 2016, now abandoned.

(30) Foreign Application Priority Data
Dec. 13, 2013 (EP) 13197157.4
Feb. 24, 2014 (EP) 14156283.5

(51) Int. Cl. A61K 31/4188; A61K 31/11; A61K 31/085; A61K 31/05; A61K 9/00; A23K 20/174; A23K 20/10; A23K 20/158; A23K 20/111; A23K 20/105; A23K 50/10

(52) U.S. Cl. CPC A61K 31/4188; A61K 31/11; A61K 31/085; A61K 31/05; A61K 9/0056; A23K 50/10; A23K 20/10; A23K 20/158; A23K 20/111; A23K 20/105; A23K 20/174

(57) ABSTRACT

The present invention is made for the purpose of improving beef quality and upgrading its fat marbling standard (which is called beef marbling standard, or BMS). The fat marbling state is generally called “Shimofuri” in Japanese. This purpose is achieved by feeding to the animal biotin in combination with a mixture of at least two essential oil compounds selected from the group consisting of thymol, eugenol, meta-cresol, vanillin and guaiacol. In a preferred embodiment of the invention, biotin is used in an amount sufficient to provide a daily dosage of 0.01 mg to 0.3 mg per kg body weight and the essential oils are administered in amounts (total dosage ranges of essential oils) of 0.5 to 4 mg per kg body weight per day. In terms of the quality of the meat, the combined use of biotin and the essential oils as defined above shows significant differences in the fat marbling (Shimofuri), luster, firmness and texture of the meat compared to the meat of non-treated animals.

### CROSS-REFERENCE

This application is a continuation of copending U.S. application Ser. No. 15/102,967, filed Jun. 9, 2016 (now abandoned), which is the U.S. national phase of International Application No. PCT/EP2014/077404 filed Dec. 11, 2014, which designated the U.S. and claims priority to EP Patent Application Nos. 13197157.4 filed Dec. 13, 2013, and 14156283.5 filed Feb. 24, 2014, the entire contents of each of which are hereby incorporated by reference.

### TECHNICAL FIELD

The present invention relates to a method of improving beef quality and upgrading its fat marbling standard (which is called beef marbling standard, or BMS). The fat marbling state is generally called “Shimofuri” in Japanese.

### BACKGROUND ART

The small flecks of intramuscular fat or marbling present in the high-priced cuts (loins and ribs) of beef and pork have long been known to influence the eating quality of meat.

In Japan for example, the content and distribution of intra-muscular fat as well as fat quality (softness and color) are important characteristics of the beef grading system and reflect consumer preferences. The fatty acid composition has been shown to play an important role in this quality attributes.

Standards exist for marbling in beef or pork carcasses. These standards are designed for use in research studies, for carcass grading, and for consumers who wish to make more informed purchasing decisions about meat.

The degree of visible marbling is described in terms of the approximate percentage of intramuscular chemical fat present as follows:
devoid 1.0-2.5%
trace 2.5-4.0%
slight 4.0-5.0%
small 5.0-6.5%
modest 6.5-7.5%
moderate 7.5-8.5%
abundant >8.5%

For research studies and carcass grading procedures, marbling must be assessed under standardized conditions. One standard method for the assessment of marbling is described in “Marbling Standards for beef and pork carcasses, S. D. M. Jones et al., Agriculture Canada Publication, 1992”.

Although fat-marbled beef (i.e. beef with an intermingling or dispersion of fat within it) is in great demand especially in Japan because of its tenderness, fine texture, etc., the problem is that it is difficult to produce. Therefore, in recent years, research in the production of the fat-marbled beef has been conducted not only in Japan but also overseas.

The current variation in marbling for beef carcasses occurs as about 1% devoid, 20% trace, 60% slight, and 19% small or greater. The preferred standards might be different dependent on regions and market needs. In Canada for example, most high-priced meat cuts sold at retail have slight marbling.

Many proposals to improve beef quality have been presented so far. For example, the Japanese patent application H6-22704 describes a method to improve the quality grade of beef, its fat marbling, firmness, flavor, tenderness, etc. by supplying feed containing calcium salt of fatty acid having a specific composition to cattle. Another method described in the Japanese patent application H06-169726 is to apply water-soluble magnesium oxide and metal chlorophyllin to cattle. By this method, when the meat is cooled during the processing of dressed carcasses after slaughter, myoglobin turns back into oxidized myoglobin by the oxygen remaining in its tissues, thus having good effects on the meat color, luster, etc. According to the Japanese patent application H07-132050, a daily intake of turmeric powder of approximately 0.1 g to 0.4 g per 10 kg of weight can prevent liver abscesses and its complications such as hepatization and liver congestion, which are frequently found in commercial cattle from the later raising stage to the fattening stage. With this improvement of hepatic function, the quality of the meat can be also enhanced. The Japanese patent application H10-113129 describes feed composed of metallic salt of fatty acid including unsaturated fatty acid and phosphorus compound optionally with antioxidant. This feed greatly contributes to improving the quality of beef, the gaining of body weight, etc. in a variety of breeds such as Japanese Black Cattle. In the Japanese patent application H11-196776, supplying feed mixed with spices and vitamin E enables the production of beef which can be kept fresh for a long time as well as reduce its drip and thus can lead to its improved flavor.

The present invention provides a new method of improving beef quality and upgrading its fat marbling standard, differently from those mentioned above.

### DESCRIPTION OF THE INVENTION

The inventors of the present application surprisingly found that the compounds as defined below, especially combinations thereof have a great potential for use in feed for animals of the subfamily Bovinae for improving beef quality, in particular for increasing intra-muscular fat deposition and marbling, i.e. for improving luster, firmness and texture of meat.

In particular, it has been found that compositions containing biotin in combination with a mixture of at least two essential oil compounds selected from the group consisting of thymol, eugenol, meta-cresol, vanillin and guaiacol have a significant effect in improving beef quality.

Therefore, the present invention relates to the use of biotin in combination with a mixture of at least two essential oil compounds selected from the group consisting of thymol, eugenol, meta-cresol, vanillin and guaiacol in feed for animals of the subfamily Bovinae for the improvement of beef quality and upgrading its fat marbling standard.

In the present context, an animal of the subfamily Bovinae (also called bovines or bovine animals) means an animal of the kingdom of Animalia, the phylum of Chordata, the class of Mammalia, the order of Artiodactyla, and the family of Bovidae. For the present purposes, Domestic cattle are the most preferred species. For the present purposes the term includes all races of domestic cattle, and all production kinds of cattle, in particular beef cattle.

The present invention also relates to a novel feed composition for animals, especially beef cattle, comprising as active ingredient biotin in combination with at least two essential oil compounds as defined above.

More particularly, this invention relates to the use of a feed composition as a component of animal feed or drinking water or feed or drinking water additives, as well as to compositions, feed additives, drinking water and feed containing them.

The term feed or feed composition means any compound, preparation, mixture, or composition suitable for, or intended for intake by an animal.

Biotin can occur in eight different stereoisomeric forms and is a known active ingredient, which can be found in numerous pharmaceutical compositions. Biotin is commercially available (for example as Rovimix® Biotin, supplied by DSM Nutritional Products, Kaiseraugst, Switzerland) or can be prepared by a skilled person using processes and methods well-known in the prior art.

The essential oil compounds according to the invention are commercially available or can be prepared by a skilled person using processes and methods well-known in the prior art.

The essential oil compounds can be used in highly purified forms in mixtures or in the form of natural available plant extracts or extract-mixtures.

The term “extract” as used herein includes compositions obtained by solvent extraction (which are also known as “extracted oils”), steam distillation (which are also known as “essential oils”) or other methods known to the skilled person. Suitable extraction solvents include alcohols such as ethanol.

By the expression “natural” is in this context understood a substance which consists of compounds occurring in nature and obtained from natural products or through synthesis. The natural substance may preferably contain at least two of the compounds as defined above as main ingredient and additionally other essential oil compounds as for example capsaicin, tannin or carvacrol.

The present invention also relates to the method for improving beef quality and upgrading its fat marbling standard, which comprises providing to the animal an effective amount of biotin in combination with a mixture of at least two essential oil compounds selected from the group consisting of thymol, eugenol, meta-cresol, vanillin and guaiacol during a period of up to 150 days before slaughtering.

In a preferred embodiment of a feeding concept, biotin being used in an amount sufficient to provide a daily dosage of 0.01 mg per kg body weight to 0.3 mg per kg body weight, preferably 0.05 mg per kg body weight to 0.2 mg per kg body weight, of the subject to which it is to be administered.

It is at present contemplated that the essential oils are administered in amounts (total dosage ranges of essential oils) of 0.5 to 4 mg per kg body weight per day, preferably 1.5 mg to 2 mg per kg body weight per day.

In another preferred embodiment of the invention the essential oils are added to the feed as a single feed additive composition.

The feed additive composition containing the essential oils according to the invention may optionally contain in minor amounts other chemical compounds, for example at least one compound found in plants, and selected from the following group, as, per kg of feed:

up to about 1 mg of propylidene, butylidene, phtalides, gingerol, lavender oil;

up to about 2 mg of deca-, undeca-, dodecalactones, ionones, irone, eucalyptol, menthol, peppermint oil, alpha-pinene;

up to about 3 mg of limonene, anethol, linalool, methyl dihydrojasmonate;

up to about 4 mg of carvacrol, propionic, acetic or butyric acid, rosemary oil, clove oil, geraniol, terpineol, citronellol;

up to about 5 mg of amyl and/or benzyl salicylate, cinnamaldehyde, a plant polyphenol (tannin);

and up to about 5 mg of a powder of turmeric or of an extract of curcuma .

All the essential oils and the additional compounds may be used in combination with an emulsifying surfactant.

The emulsifying agent can be selected advantageously from those of a rather hydrophilic nature, for example among polyglycerol esters of fatty acids such as esterified ricinoleic acid or propylene glycol esters of fatty acids, saccharo-esters or saccharo-glycerides, polyethylene glycol, lecithins etc.

Examples of particularly preferred dosages of the essential oil compounds in a final feed additive composition according to the invention are independently from each other in the following ranges:

thymol between 80 and 120 g/kg, preferably 101 g/kg;

eugenol between 20 and 60 g/kg, preferably 30 g/kg;

meta-cresol 80 and 110 g/kg, preferably 90 g/kg;

vanillin between 30 and 70 g/kg, preferably 50 g/kg

guaiacol between 20 and 50 g/kg, preferably 35 g/kg

salicylate between 10 and 30 g/kg, preferably 25 g/kg

resorcine between 5 and 20 g/kg, preferably 15 g/kg

In a preferred embodiment of a feeding concept the final feed includes biotin and a mixture of thymol, meta-cresol and vanillin, wherein these four compounds being used in amounts sufficient to provide a daily dosage of 0.05 mg to about 0.2 mg biotin and 1.5 mg to 2 mg total essential oils per kg body weight of the subject to which it is to be administered.

The incorporation of the composition of feed additives as exemplified herein above to animal feeds is in practice carried out using a concentrate or a premix. A premix designates a preferably uniform mixture of one or more micro-ingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

A premix may comprise 2-10% by weight of the active mixture or natural substance or extract, 0-40% by weight of other conventional additives, such as flavorings, and 50-98% by weight of any conventional absorbing support.

The support may contain, for example, 40-50% by weight of wood fibers, 8-10% by weight of stearine, 4-5% by weight of curcuma powder, 4-5% by weight of rosemary powder, 22-28% by weight of limestone, 1-3% by weight of a gum, such as gumarabic, 5-50% by weight of sugar and/or starch and 5-15% by weight of water.

This premix is then mixed with vitamins, mineral salts and other feed additive ingredients and then finally added to the feed.

The term “feed” as used herein includes biotin and a feed additive composition according to the invention and other components. A typical cattle feed comprises as other components 60% to 90% corn or corn silage, between 3% and 10% SBM (Soya bean meal), 1% to 2% minerals and optionally between 5% and 20% hay, cereals and straw.

1. A method of improving fat marbling standard of animal meat comprising administering to an animal an amount sufficient to provide a daily dosage of 0.01 mg to 0.3 mg per kg body weight of the animal of biotin in combination with an amount sufficient to provide 0.5 mg to 4 mg per kg body weight per day of the animal of a mixture of at least two essential oil compounds selected from the group consisting of thymol, eugenol, meta-cresol, vanillin and guaiacol so as to improve fat marbling standard of meat from the animal.

2. The method according to claim 1, which comprises administering the biotin in an amount sufficient to provide a daily dosage of 0.05 mg to 0.2 mg per kg body weight of the animal, and administering the mixture of at least two essential oils in an amount of 1.5 mg to 2 mg per kg body weight per day of the animal.

3. The method according to claim 1, wherein the biotin and the mixture of the at least two essential oils are administered in sufficient amounts to improve luster, firmness and texture of the animal meat.

4. The method according to claim 1, wherein the biotin and the mixture of the at least two essential oil compounds are administered to the animal during a period of up to 150 days before slaughtering.

5. The method according to claim 1, wherein the animal is domestic beef cattle.

6. An animal feed additive composition for improving fat marbling standard of animal meat comprising an amount sufficient to provide a daily dosage of 0.01 mg to 0.3 mg per kg body weight of the animal of biotin in combination with an amount sufficient to provide a daily dosage of 0.5 mg to 4 mg per kg body weight per day of the animal of a mixture of at least two essential oil compounds selected from the group consisting of thymol, eugenol, meta-cresol, vanillin and guaiacol, the biotin and the mixture of at least two essential oils being present in amounts to improve fat marbling standard of the meat of the animal.

7. The animal feed additive composition according to claim 6, wherein the biotin is present in an amount sufficient to provide a daily dosage of 0.05 mg to 0.2 mg per kg body weight of the animal, and the mixture of at least two essential oils is present in an amount sufficient to provide a daily dosage of 1.5 mg to 2 mg per kg body weight per day of the animal.
